(19) United States
(12) Patent Application Publication
(10) Pub. No.: US 2005/0272951 A1
(43) Pub. Date: Dec. 8, 2005

(54) Process for the separation of the water produced in the catalytic oxidation of aromatic hydrocarbons to polycarboxylic aromatic acids

(76) Inventor: Sergio Noe', Milan (IT)

Correspondence Address: James V Costigan;Hedman & Costigan, 1185 Avenue of the Americas, New York, NY, 10036-2601, US

(21) Appl. No.: 10/518,070

(22) PCT Filed: May 30, 2003
(86) PCT No.: PCT/EP03/05786

§ 371 Date: Jul. 25, 2005

(30) Foreign Application Priority Data
Jun. 27, 2002 (IT) 2002A001423

(51) Int. Cl. C07C 51/255

(52) U.S. Cl. 562/412

(57) ABSTRACT

In the oxidation process of aromatic hydrocarbons to produce the corresponding aromatic acids and anhydrides, such as isophthalic and terephthalic acid and their homologous products, by catalytic oxidation in the presence of acetic acid as reaction medium, the water, by-product of the oxidation reaction, is separated from the acetic acid to be recycled by means of azeotropic/extractive distillation, using, as modifying solvent of their relative volatility, the same aromatic hydrocarbon fed to the oxidation reaction.

The present invention relates to a process for the production of polycarboxylic aromatic acids with particular reference to the separation step from the reaction medium of water, which is the by-product, and recovery of the reaction solvent to be recycled.

In the petrochemical industry, polycarboxylic aromatic acids form a class of intermediate compounds of considerable importance for the production of plastic materials and resins with high chemical and mechanical characteristics, high-quality synthetic fibers for technical articles, furnishing and clothing, as well as for the production of additives and behavioural products, such as, for example, plasticizers, additives for lubricants, dyes, pharmaceutical products and cosmetics.

This class of polycarboxylic aromatic acids includes, inter alia, terephthalic acid which is industrially obtained from the oxidation of para-xylene, isophthalic acid from the oxidation of meta-xylene, or thophthalic acid from the oxidation of ortho-xylene, trimellitic anhydride from the oxidation of pseudo-cumene and so on for homologous products such as mesitylene and durene.

The oxidation reaction of aromatic hydrocarbons is normally carried out in these processes, according to the most recent and commercially successful technologies, by catalytic oxidation with atmospheric oxygen, in liquid phase and with homogeneous catalysts, using acetic acid as reaction medium. For example, with reference to the production of terephthalic acid from para-xylene, the reaction takes place as follows:

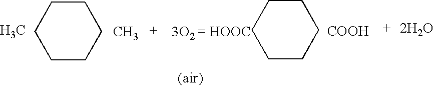

As can be easily observed, for each mole of terephthalic acid, which is the desired product, two moles of water are also obtained as by-product, and these must be separated and suitably disposed of, whereas the acetic acid, which represents the reaction medium, is recovered according to specification and recycled to the reaction. The technical problem to be solved with the present invention therefore relates to the separation and disposal of the water by-product.

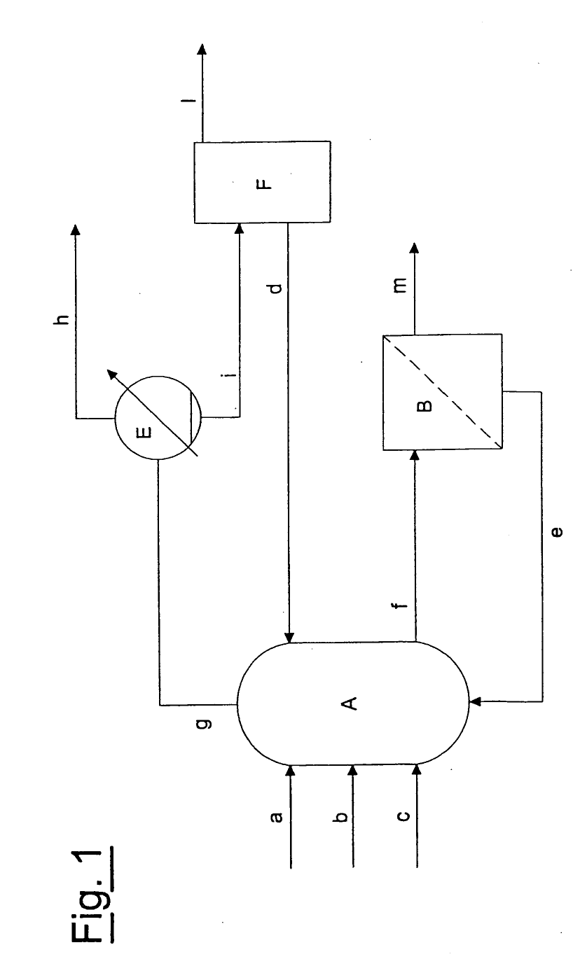

In order to better illustrate the technical problem faced with the process according to the invention, in the following description reference is made to the oxidation reaction of para-xylene to produce terephthalic acid, which represents a typical and commercially important case; it should be explicitly pointed out however that the process for the treatment of the reaction product to separate water and recycle acetic acid, can be advantageously used in oxidation reactions of other aromatic hydrocarbons, effected with the same method, i.e. carried out in liquid acetic acid as reaction medium. In order to expound the technical problems faced and the advantages of the process according to the present invention, FIG. 1 illustrates the overall process scheme for the production of terephthalic acid from para-xylene, which comprises a reaction section, a product recovery and purification section and a treatment and recovery section of acetic acid, separating and discharging the water produced in the reaction.

In general, the production of aromatic acids is based, as already mentioned, on the oxidation of the corresponding alkyl aromatic compounds with gaseous oxygen, using air as the preferred source of oxygen, in the presence of a homogeneous catalyst. For example, patent application WO 98/29378 describes and claims an oxidation process of this type, with a catalytic system operating in a liquid, homogeneous phase.

The oxidation reaction proceeds from left to right, also favoured by the fact that polycarboxylic aromatic acid normally has a low solubility in acetic acid. The polycarboxylic aromatic acid can therefore be easily separated from the reaction medium by the conventional liquid/solid separation means, such as filtration and centrifugation. The heat developed by the reaction is discharged by partial evaporation of the reaction medium, which is specifically aqueous acetic acid. This is the technique which is most widely-used in the most recent industrial plants, due to its economical and plant-engineering advantages.

It is immediately evident that water, the by-product which is to be separated and disposed of, is more volatile than acetic acid and therefore passes more easily into vapour phase. The vapour developing from the reaction medium is consequently richer in water than the acetic acid solution in which the reaction takes place and from which it is released by evaporation. In industrial practice, the oxidation reactor operates under temperature and pressure conditions which maintain it under stationary conditions, whereas the reaction water is discharged from the reactor as vapour, together with the acetic acid corresponding to the thermodynamic equilibrium. The gaseous phase developing from the reactor also contains the incondensable phase consisting of the exhausted air.

The block scheme of FIG. 1 illustrates the general oxidation process with air of alkyl aromatic hydrocarbons, with particular reference, for the sake of convenience, to the case of terephthalic acid.

Para-xylene, air and the oxidation catalyst are fed into the reactor A, through line a, line b and line c, respectively. The acetic acid separated from the water produced in the reaction, also returns to the reactor A through line d, as well as the mother liquor resulting from the separation of terephthalic acid, through line e.

The reaction conditions vary according to the catalytic system used and, generally speaking, temperatures within the range of 115-220° C. and relative pressures of 2-25 bar (0.2-2.5 Mpascal), are adopted. The water content in the reaction medium is maintained within the range of 3-10% by weight with respect to the acetic acid.

In general, the reaction product, the raw terephthalic acid, is extracted in continuous, as a suspension at 15-25% by weight, from the reactor A through line f and sent to the separation unit B, from which a residue of raw terephthalic acid is obtained, to be sent, by means of line m, to the subsequent steps, not shown in the figure, for the final purification. The mother liquor is recovered from unit B, and essentially consists of a catalyst solution in acetic acid and the intermediate oxidation products, such as p-toluic acid and 4-carboxybenzaldehyde, which is recycled to the reaction through line e.

The exhausted air is discharged from the reactor A through line g and is saturated with acetic acid and water, thus removing both the reaction heat and the whole water formed during the partial oxidation of p-xylene to terephthalic acid. This stream is sent to the condenser E, which separates a liquid phase of aqueous acetic acid from the exhausted air, which is discharged through line h and undergoes suitable abatement operations, not shown in the figure, to allow it to be discharged into the atmosphere according to the regulations.

The water/acetic acid solution is sent, through line i, to the separation plant F in which the water, produced in the reaction and to be disposed of, is separated from the dehydrated acetic acid which must be recycled. The stream of line i fed to the dehydration unit F has a water content ranging from 7 to 30% by weight, according to the oxidation process carried out in the reactor A.

The separation plant F has the function of separating the amount of water produced in the reaction in unit A, discharging it with a very low amount of acetic acid, typically lower than 2000 ppm, in order to reduce the reintegration cost of the latter. The purified water is sent, through line l, to the final decontaminating treatment, not shown in the figure. The amount of water allowed in the acetic acid recycled from unit F to the reactor A through line d must not exceed 2-4% by weight.

The present invention specifically relates to the separation section F of the water by-produced and the recycling of the acetic acid, by processing the stream of line i of the scheme of FIG. 1 and returning the acetic acid dehydrated up to 2-4% through line d.

A substantial stream of aqueous acetic acid, at a flow rate in the order of hundreds of cubic meters per day, with a water content ranging from 7 to 30% by weight, more often about 10%, consequently reaches section F for the recovery of the acetic acid and separation of the water by-produced. The separation of the acetic acid from the reaction water proves to be intrinsically extremely onerous for the following reasons.

The thermodynamic characteristics of the water/acetic acid binary system, relating to their liquid/vapour equilibrium, are such as to require high heat and refrigeration consumptions in order to separate, by distillation, the two substances with an acceptable degree of purity. If it is considered the liquid-vapour equilibrium curve of this binary system, their relative volatility value has a very unfavourable trend. The relative volatility, as is known, expresses the easiness, or difficulty, in separating two substances by distillation: the relative volatility α₁₂ of two compounds having indexes 1 and 2, expresses the ratio Y₁ x₂/x₁y₂ wherein:
y₁ is the concentration of 1 in vapour phase
x₂ is the concentration of 2 in liquid phase
x₁ is the concentration of 1 in liquid phase
y₂ is the concentration of 1 in vapour phase

The volatility α₁₂ of water (suffix 1) with respect to the acetic acid (suffix 2), under the approximately atmospheric pressure conditions normally adopted, has a value of approx. 2 when the water concentration is low, it then drops to 1.6÷1.7 within the range of 94-95% molar of water, and subsequently tends to be 1 when the concentration is close to 100% of water. With these trends of the relative volatility, it is relatively easy to obtain acetic acid, at the bottom of the distillation column, at a purity degree acceptable for being recycled to the oxidation step of p-xylene. It is, on the contrary, extremely onerous to obtain, at the head of the distillation column, water with an acetic acid content corresponding to the environmental specifications in force and, in any case, for the process economy, it is necessary to reduce the reintegration of acetic acid to the minimum, due to the losses in the resulting water. The presence of the acetic ion, moreover, in the resulting water requires costly purifications before being discharged into the environment.

For these two reasons, the purity degree of the water obtained by distillation of the acetic acid/water solution must be very high and close to 100%, thus requiring a large number of separation steps, or “equivalent” trays in the column, and a high reflux at the head, thus requiring a large consumption of vapour in the bottom reboiler and refrigeration in the head condenser. This situation has stimulated research for more sophisticated separation processes with respect to conventional distillation, in order to limit energy consumption.

One of the alternative treatment techniques proposed in the state of the art is azeotropic distillation, using, for example, isobutyl acetate as third component. Liquid-liquid extraction is another technique proposed, adopting a solvent having a low solubility with water. This technology can obtain water purified from acetic acid, but it is extremely difficult to obtain, on the other side, an acetic acid with a low water content, which can be recycled directly to the reaction. According to the known technique, resort must therefore be made to costly and sophisticated solvents, such as alkyd derivatives of oxidized phosphine for the recovery of the last part of acetic acid from the water, if acetic acid is to be obtained within specification, also on the other side.

These alternative technologies for the treatment of the water—acetic acid mixture, achieve the objective of reducing the energy consumptions, but create other problems due to their complexity and non-negligible costs. The extraction solvent is costly and generally polluting, like acetic acid or even more: the solvent must therefore be recovered from all the liquid and gaseous streams leaving the plant or which are recycled to the reaction. The plant is consequently considerably penalized as far as investment, running costs, energy consumption and labour are concerned. The cost necessary for the reintegration of the solvent lost or degraded is also high.

An objective of the present invention is to provide a treatment process of the mixture of water—acetic acid which recovers the acetic acid with a high yield and with a purity degree suitable for allowing it to be recycled to the oxidation step and which separates the water by-produced at a high purity, avoiding the drawbacks of the systems of the known art.

This objective, according to the present invention, is achieved by means of the process according to the most general definition of claim 1 and for the preferred embodiments or possible variations defined in the dependant claims.

The characteristics and advantages of the separation process of water from acetic acid according to the present invention will appear more evident from the following illustrative but non-limiting description, referring to its application to the oxidation of p-xylene to produce terephthalic acid, which represents a typical and industrially important case.

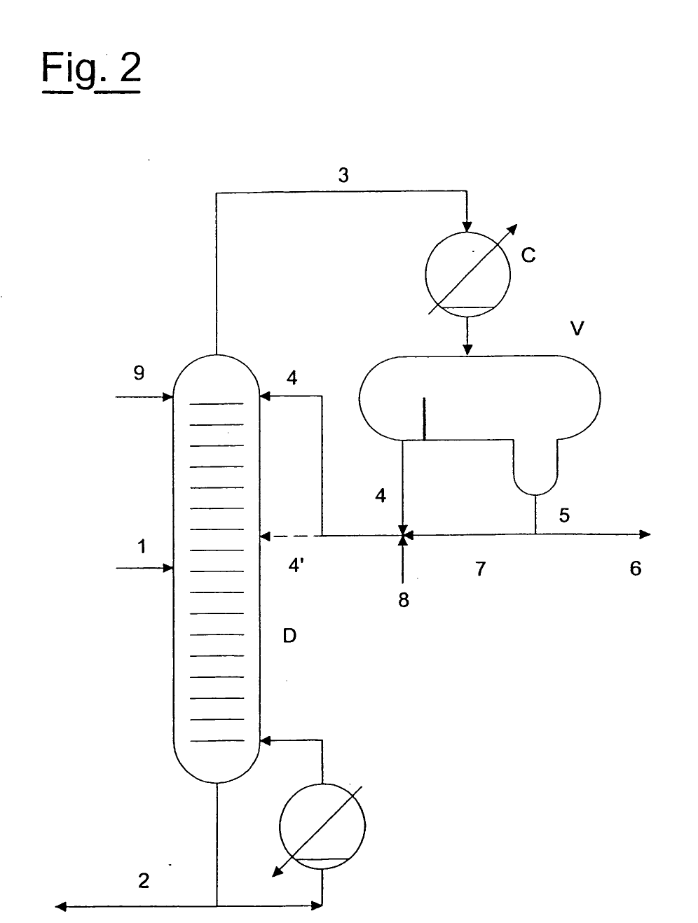

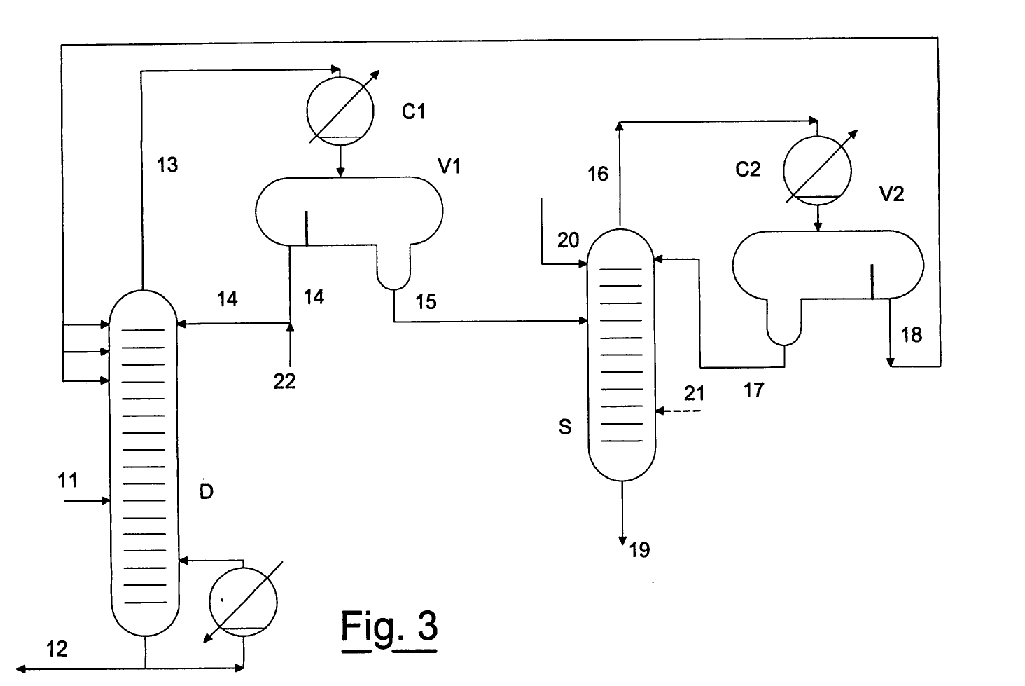

FIG. 1 shows the general block scheme of the overall process. FIG. 2 shows the process embodiment according to the invention, in the section for the recovery of the acetic acid and separation of the water as by-product from the substantial stream of aqueous acetic acid which arrives from the reaction unit upstream, as illustrated above with reference to FIG. 1.

In the illustrative embodiment of FIG. 2, the separation of water from acetic acid is effected in a single distillation column D, obtaining water and acetic acid regenerated under conditions compatible with the process requirements and environmental specifications. The aqueous acetic acid coming from the oxidation step is introduced into the column through line 1. Line 1 coincides with line i of the block scheme of FIG. 1. Acetic acid and the extraction solvent fed to the upper part of column D are obtained with the bottom stream of line 2. The extraction solvent typically consists of the same hydrocarbon to be fed as raw material to the oxidation reaction, in this case p-xylene. The stream of line 2 can therefore be fed directly to the oxidation reaction. Line 2 coincides with line d of the block scheme of FIG. 1.

The head product—consisting of vapours having a composition corresponding to the heterogeneous azeotropic water/hydrocarbon mixture (in our case water/p-xylene), which, condensed in the condenser C and de-mixed in the vessel V, separate into the two liquid phases which characterize the azeotropic mixture—is collected by means of line 3 from the head of column D. The organic phase is collected by means of line 4 and completely resent to column D, the heavier water phase is discharged with line 5 and can be totally sent to the water treatment through line 6 or, alternatively, partially resent to the column through line 7, together with the stream of line 4, for a better abatement of the acetic acid.

The hydrocarbon to be oxidized (in this illustrative case p-xylene) and subsequently fed to the reaction together with the acetic acid of the bottom of the column, is fed to the head of the column, either separately through line 9 or, through line 8, together with one or more of the streams, the organic stream of line 4 and the aqueous stream of line 7, which converge into line 8 and which are refluxed to the column, together or separately.

The fresh feeding stream of p-xylene in the column D is compatible with the material balance of the oxidation reactor, i.e. the quantity of p-xylene necessary as extraction solvent for the separation between water and acetic acid is lower than—or, at most, equal to—the quantity of p-xylene corresponding to the oxidation capacity of the plant. The fresh p-xylene which is fed to the separation section before being used in the oxidation reaction, can be the whole amount of fresh p-xylene or only a part of it, approximately from 40 to 100%, whereas the complement to 100% can be introduced directly to the reaction step.

In the scheme of FIG. 2, the hydrocarbon stream is refluxed to the head of column D through line 10: according to an embodiment variation of the process, it can be fed—completely or partially—according to the scheme shown in FIG. 2, to a few trays lower than the feeding of the fresh hydrocarbon from line 9, through line 11 indicated with a dotted line.

In the same way, the aqueous stream refluxed into the column through lines 5,7, can be distributed at several heights of column D, for example between line 10 and 11.

The condensation and cooling action necessary for the separations carried out in column D, is effected—in FIG. 2—by the condenser C, situated outside the column itself. As an alternative, this action can be profitably integrated by inducing additional cooling steps inside the column D. This effect can be obtained by using dephlegmators, i.e. cooling exchangers positioned inside the upper part of column D, preferably a few trays below its top. Similarly, this effect can be obtained by under-cooling the reflux liquid streams of lines 9,10,11.

### EXAMPLE

Using the scheme of FIG. 2, 1000 kg/h of aqueous acetic acid, with a water content of 20% by weight, are fed in continuous to column D, operating at atmospheric pressure and equipped with 60 bubble cup trays. The feeding is effected at the 50^{th} tray starting from the head of the column. 507 kg/h of p-xylene are introduced onto the head tray. The head vapours are condensed by cooling with water and the two resulting liquid phases are collected and separated in a gravity separator.

The organic phase is totally re-introduced onto the third tray of the head section, together with 40% of the aqueous phase. The remaining 60% of the aqueous phase is extracted from the cycle as a head product and contains 2060 ppm of acetic acid and 945 ppm of p-xylene. These amounts require decontaminating treatment, of the biological type, for example, before discharging the aqueous phase thus separated, into the environment. The product of the bottom of the column consists of a stream of acetic acid with 1.52% by weight of water and 32.83% by weight of p-xylene: it is directly fed to the oxidation reactor. The head temperature is maintained at 92.5° C. and the temperature at the bottom is 130° C.

The separation process of water from acetic acid, according to the present invention, allows considerable advantages with respect to the processes of the known art, as far as the complexity and investment of the plant are concerned as also its energy costs. These advantages are basically due to the fact that the process according to the invention does not introduce any component not inherent to the oxidation process, into the separation process: the extraction solvent consists of the same raw material fed to the oxidation.

In the oxidation process which produces terephthalic acid starting from p-xylene, it is specifically p-xylene which is used as extraction solvent in the separation of water from acetic acid. The same also occurs in the process relating to isophthalic acid, wherein meta-xylene is used as extraction solvent, and so on.

This allows considerable advantages. First of all, the presence of a hydrocarbon much more similar to acetic acid than to water, causes a considerable increase in the volatility α₁₂ of the water with respect to acetic acid. Acetic acid and the aromatic hydrocarbons in question are preferably miscible in all proportions, whereas water and hydrocarbons have a very narrow range of reciprocal solubility. They form minimum heterogeneous azeotropic mixtures, in which the α₁₂ value reaches values of several tens of units. Also under the conditions prevailing at the bottom of column D, with the presence of the hydrocarbon—p-xylene, o-xylene, m-xylene and so on—the relative volatility α₁₂ is increased to more than double the value with respect to the pure system of acetic acid/water. The recycled acetic acid consequently has a small amount of water under the same conditions. With the same quantity of residual water in the acetic acid leaving the column D and recycled to the oxidation reactor, an energy saving is, in the other hand, obtained, both with respect to the heat in the reboiler and cooling means in the condenser, accompanied by a lower overall investment.

All problems present in the processes of the known art, which use foreign substances as extraction agents, relating to pollution, recovery, losses and reintegration of the substances used in the cycle as extraction agents, are thus avoided.

1. An oxidation process of aromatic hydrocarbons, such as ortho-, meta- and para-xylene, pseudocumene, mesitylene and their homologous products, to produce the corresponding acids and anhydrides, such as orthophthalic acid, isophthalic acid and terephthalic acid, trimellitic anhydride, trimesic acid, respectively, and their homologous products, by catalytic oxidation with acetic acid as reaction medium, characterized in that the water which forms the by-product of the oxidation reaction is separated from the acetic acid, containing it in the effluent produced by the reaction, by means of azeotropic/extractive distillation, using as extraction solvent, modifier of the relative volatility of said components, the same aromatic hydrocarbon which is fed to the oxidation reaction, obtaining acetic acid as bottom distillation stream, to be recycled to the reaction together with the extraction solvent.

2. The oxidation process of aromatic hydrocarbons according to claim 1, characterized in that the hydrocarbon which is fed to the separation section of water from acetic acid forms a part of the overall hydrocarbon fed to the oxidation plant.

3. The oxidation process of aromatic hydrocarbons according to claim 2, characterized in that the hydrocarbon which is fed to the separation section of water from acetic acid forms 40-100% of the overall hydrocarbon fed to the oxidation plant.

4. The oxidation process of aromatic hydrocarbons according to claim 1, characterized in that the reaction water is separated as head product from the acetic acid in a column (D), in which the hydrocarbon to be fed to the oxidation reaction is introduced into its upper part as azeotropic/extraction solvent, through line (9), obtaining from the head of the column (D), through line (3), vapours of the water/hydrocarbon azeotropic mixture of which, once condensed in the condenser (C) and demixed in the vessel (V), the organic phase is totally refluxed to the column (D) with line (4), the heavier aqueous phase is discharged with line (5), the stream of the bottom of the column (D) discharged with line (2) containing acetic acid together with the azeotropic/extraction solvent fed to the upper part of the column (D).

5. The oxidation process of aromatic hydrocarbons according to claim 4, characterized in that the aqueous phase of line (5) is partially refluxed to the column (D) with line (7).

6. The oxidation process of aromatic hydrocarbons according to claim 4, characterized in that the hydrocarbon stream refluxed to the head of the column (D) is fed either totally or partially—to a few trays lower than the feeding of the fresh hydrocarbon of line (9), through line (4′).

7. The oxidation process of aromatic hydrocarbons according to claim 5, characterized in that the aqueous stream refluxed to the column (D) is distributed at various heights.

8. The oxidation process of aromatic hydrocarbons according to claim 4, characterized in that the hydrocarbon forming the azeotropic/extraction solvent is fed to the column (D) through line (8), together with the refluxed stream of line (4), converging therein.
